(19) United States
(12) Patent Application Publication
(10) Pub. No.: US 2025/0034206 A1
(43) Pub. Date: Jan. 30, 2025

(54) METHOD OF PROTEIN PURIFICATION

(71) Applicant: FERRING B.V., Hoofddorp (NL)

(72) Inventors: Eyal KAMHI, Kiryat Malachi (IL); Michelle MINTZ, Kiryat Malachi (IL); Jenny AHARONOV, Kiryat Malachi (IL); Elinor EREZ, Kiryat Malachi (IL)

(73) Assignee: FERRING B.V., Hoofddorp (NL)

(21) Appl. No.: 18/717,177

(22) PCT Filed: Dec. 7, 2022
(86) PCT No.: PCT/EP2022/084766

§ 371 Date: Jun. 6, 2024

(30) Foreign Application Priority Data
Dec. 8, 2021 (EP) 21213254.2

(51) Int. Cl. C07K 1/22

(52) U.S. Cl. CPC C07K 1/22

(57) ABSTRACT

The present invention provides methods for use in protein purification. The disclosed methods help avoid precipitation events and can be used to improve the performance and/or efficiency of protein purification protocols. The methods may be used to improve or prepare solutions for use in protein purification and/or protein extraction processes. One particular application of the disclosed method is in affinity chromatography where it may be applied to the preparation of load solutions.

### FIELD

The present invention provides methods for use in protein purification. The disclosed methods help avoid precipitation events and can be used to improve the performance and/or efficiency of protein purification protocols. The methods may find particular application in affinity chromatography.

### BACKGROUND

Proteins and peptides for use in medicine are often prepared from recombinant systems. In such cases, the useful protein or peptides often need to be purified and/or extracted from complex mixtures. Sensitive and reliable methods of purification are required to ensure that target proteins and peptides can be purified to a suitable yield and purity.

Specific (or target) proteins or peptides can be purified using any number of different and well-known techniques. Affinity chromatography represents a particularly accurate, reliable and sensitive method of extracting, to a high degree of purity, a particular target protein/peptide. However, the efficiency and performance of these techniques is easily upset by precipitation events.

The present disclosure aims to provide methods and techniques that can be used to minimise those precipitation events and in turn improve the yield and purity of purified proteins.

### SUMMARY

The present disclosure provides methods that may improve or prepare solutions for use in protein purification and/or protein extraction processes.

For the avoidance of doubt, a solution may contain a solvent and a solute; the solute component may comprise one or more proteins or peptides. Accordingly, the methods described herein may be applied to solutions that contain protein(s) or peptide(s).

For convenience and as used herein, the term “protein” shall be taken to also embrace shorter molecules that might otherwise be referred to as “peptides”. Accordingly, a solution comprising a protein may, for example, comprise one or more protein(s) and/or peptide(s).

Solutions, particularly those that contain protein(s), are susceptible or prone to precipitation events. For example, precipitation may occur as the solvation potential of the solvent component of the solution is altered. Without wishing to be bound by theory, alteration of the solvation potential may lower the solubility of the solute (for example a protein and/or peptide) resulting in some precipitation of the solute.

The addition of one or more reagents to a solution may alter the solvation potential of a solution. Additionally or alternatively, changes to the pH, temperature and/or composition of a solution may also alter its solvation potential.

In view of the above, the precipitation of a protein from a solution may occur in response to the addition of a reagent and/or changes to the pH, temperature and/or composition of the solution.

The methods described herein may be applied to solutions that contain one or more protein(s) for purification or extraction. Hereinafter, the term “purification” will be used to describe all protein/peptide purification and/or extraction processes/events.

One or more protein(s) for purification may be referred to as “target protein(s)”. It should be understood that the term “target protein” refers to any protein (or peptide) that is to be selectively extracted and/or purified from a solution. Any given target protein may be purified or extracted from a simple or complex mixture of other solutes, for example other protein(s). For example, one or more target protein(s) may be purified or extracted from a solution containing a homogenous or heterogeneous mix of protein(s).

The amount of target protein present in a solution may vary. For example, any of the solutions described herein (including for example any of the load solutions described below) may comprise between about 0.01 mg/ml protein and 0.50 mg/ml of any given target protein.

For example, a solution of this disclosure may comprise about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.10 mg/mL, 0.11 mg/mL, 0.12 mg/mL, 0.125 mg/ml, 0.13 mg/mL, 0.14 mg/mL, 0.15 mg/mL, 0.16 mg/mL, 0.17 mg/mL, 0.175 mg/ml, 0.18 mg/mL, 0.19 mg/mL, 0.20 mg/ml, 0.25 mg/ml, 0.25 mg/mL, 0.275 mg/mL, 0.30 mg/mL, 0.325 mg/mL, 0.35 mg/mL, 0.375 mg/mL, 0.40 mg/mL, 0.425 mg/mL, 0.45 mg/mL or about 0.475 mg/mL of any given target protein. For example, a solution of this disclosure may comprise about 0.07 to about 0.138 mg/ml of a target protein. In one teaching, a solution of this disclosure may comprise about 0.09 mg/ml of a target protein.

The total protein content of the solution (that is the total of all protein in a solution of this disclosure (including any target protein)), may also vary and may lie within the range of about 0.5 to about 2 mg/ml, for example about 0.6 mg/ml, 0.7 mg/ml, 0.8 mg/ml, 0.9 mg/m, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml or 1.9 mg/ml. The total protein content may be between about 0.7 and 1.8 mg/mL. In one embodiment, the total protein content may be 1.3 mg/ml.

Proteins may be extracted or purified from solutions (including solutions that comprise heterogeneous or homogeneous mixes of proteins) by a number of different techniques. For example proteins may be purified or extracted using size exclusion chromatography, hydrophobic interaction chromatography, ion exchange chromatography, free-flow electrophoresis, affinity chromatography and high performance liquid chromatography (HPLC).

By way of example, affinity chromatography is a separation technique based on the interaction between a target protein and a ligand. Typically, affinity chromatography exploits a resin or matrix coupled to a ligand specific for one or more target protein(s). The ligand may be, for example, an antibody or a lectin. Affinity purification techniques tend to be very specific but are also prone to non-specific binding events and clogging through precipitated solutes; this reduces the overall efficiency of the process and the purity of the yield.

The methods described herein can be applied to solutions that are intended to be exploited in a protein purification procedure, for example a chromatography or affinity chromatography process. For example, a solution to be subject to a method described herein may contain one or more target protein(s) and may be for loading onto an affinity chromatography column. Solutions of this type may be referred to as “load solutions”.

Load solutions, from which one or more target protein(s) are to be extracted or purified, may also require optimisation to reduce problems associated with, for example, non-specific binding (for example as might occur between non-target solutes within the load solution and a(n affinity) matrix of a chromatography column) and solute precipitation, including target protein precipitation. One of skill will appreciate that load solution optimisation might ensure maximum protein yield and purity and/or proper functioning of the chosen purification/extraction process.

For the avoidance of doubt, the term “protein yield” refers to the amount of target protein obtained from a purification or extraction procedure. Many factors affect the yield; for example the yield may be lower than expected if the load solution exhibits precipitation of one or more of the solutes.

The term “purity” relates to the level of contamination within any of the obtained fractions. A fraction containing one or more target protein(s) should contain as low a level of contamination as possible. Non-target solutes, for example non-target proteins, may represent unwanted contaminants. A pure or substantially pure fraction may contain a low or undetectable level of contamination. The methods described herein may be used to help reduce the level of contamination in any given fraction, in particular those fractions that contain the target protein(s).

In view of the above, an efficient protein purification process is one that yields a sufficient quantity of target protein purity with an acceptable level of contamination.

Solutions from which one or more target proteins are to be extracted or purified, may require optimisation before being fed into a purification procedure; any optimisation being necessary to ensure that the selected purification procedure, delivers the target protein(s) at a suitable yield and purity.

Optimisation protocols may comprise, for example, adjusting the composition and/or pH of the solution before it is fed into the selected purification procedure.

In some cases, an optimisation protocol can itself induce protein precipitation. In other words, the parameters which ensure optimal performance of a purification/extraction procedure and purity and yield are sometimes contrary to those parameters which minimise non-specific binding events and solute (including target protein) precipitation. Indeed protein precipitation sometimes occurs as a consequence of the protocols used (and which are necessary) to optimise load solutions.

The methods provided by this disclosure allow solutions that contain target proteins, to be optimised for use in purification processes but at the same time reduce or minimise the risk of that optimisation process leading to solute (i.e. target protein) precipitation.

Moreover, the methods described here allow load solutions to be optimised for maximum efficiency, purity and yield in a purification procedure, while at the same time avoiding those precipitation events associated with prior art optimisation techniques.

One of skill will appreciate that any method which allows a load solution to be optimised but which reduces the risk of solute precipitation, will help increase target protein yield, the purity of that yield and the useful life of the purification apparatus (for example a purification column).

Without wishing to be bound by theory, these advantages are brought about because the methods described herein help retain proteins in solution, this prevents apparatus, for example columns used in chromatography and affinity chromatography procedures, from becoming clogged and also reduces non-specific binding.

Accordingly, in a first aspect, there is provided a method of preventing precipitation from a solution, said method comprising contacting the solution with an amount of polysorbate.

The term ‘polysorbate’ may embrace all members of the relevant class of oily liquid emulsifiers derived from ethoxylated sorbitan (a derivative of sorbitol) esterified with fatty acids. The term polysorbate may further include polysorbate 20 (PS20), polysorbate 40 (PS40), polysorbate 60 (PS60) and polysorbate 80 (PS80). The term ‘polysorbate’ may include, for example, those compounds known under the brand names Kolliphor, Scattics, Alkest, Canarcel, and Tween.

In a yet further aspect, there is provided a method of preventing precipitation from a solution, said method comprising contacting the solution with an amount of polysorbate20 (PS20).

The solution may comprise one or more solutes.

The solutes may comprise one or more proteins.

The solutes may comprise one or more target protein(s)—namely a protein or proteins that are to be extracted or purified from the solution.

One or more of the solutes, for example one or more protein(s) (including one or more of the target protein(s)) may, under certain conditions, precipitate from the solution. The methods described herein can be used to prevent, reduce or inhibit the precipitation of solutes from solutions. Indeed the methods described herein may be used to prevent, reduce or inhibit the precipitation of solutes from solutions under those conditions which might normally be expected to induce precipitation.

The solution may be a load solution. Accordingly, the methods described here may be used to prevent precipitation from a load solution, said method comprising contacting the load solution with an amount of polysorbate, for example polysorbate 20 (PS20).

For example, the disclosed method may be used to prevent precipitation of one or more protein(s) from a load solution and/or to prevent precipitation of one or more target protein(s) from a load solution. Methods of this type might comprise contacting a load solution with an amount of polysorbate, for example, polysorbate 20 (PS20). Moreover, the methods may prevent or reduce precipitation events even when solutions, for example, load solutions, are optimised for use in a chromatography or affinity chromatography process.

However, polysorbate (including PS20) generates radicals that may increase the oxidation of the target proteins for purification. Selecting a specific polysorbate concentration and/or incubation time has now been shown to lower the risk of such oxidation events.

Polysorbate (e.g. polysorbate 20) may be added to the solution, for example the load solution, such that the final concentration of polysorbate (e.g. polysorbate 20) in the solution is between about 0.1% and 5% (% w/v). For example polysorbate (including polysorbate 20) may be added such that the final concentration of polysorbate (e.g. polysorbate 20), in the solution, is about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8% and about 4.9% (all concentrations are % w/v).

In the context of this disclosure, the term ‘about’ may mean±a final concentration of polysorbate/polysorbate20 (in the solution) of 0.05% (% w/v).

The methods described herein may use polysorbate/polysorbate 20 at a final concentration (in the solution) of 0.1%, 0.25%, 0.5%, 0.75% or 1.0% (% w/v).

Once the polysorbate (including polysorbate 20) has been added to the solution, the polysorbate or PS20/solution mix is incubated at a suitable temperature for a predetermined length of time.

The polysorbate or PS20/solution mix may be incubated for anywhere between about 1 min and 2 hours. For example, the PS20/solution mix may be incubated for about 5 minutes, about 10 minutes, about 15 minutes, about 20 minutes, about 30 minutes, about 40 minutes, about 50 minutes, about 70 minutes, about 80 minutes, about 90 minutes, about 100 minutes, about 110 minutes or about 115 minutes. In the context of the length of the incubation time, the term ‘about’ may mean±5 min. For example, the polysorbate or PS20/solution mix may be incubated for anywhere between 15 and 25 minutes (for example 20 minutes).

The polysorbate or PS20/solution mix may be incubated at a temperature anywhere between about 10° C. and 30° C. For example, the polysorbate or PS20/solution mix may be incubated at a temperature of about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C. or about 29° C. In the context of the incubation temperature, the term about may mean±2° C. The polysorbate or PS20/solution mix may be incubated at a temperature anywhere between about 21° C. and 25° C. (for example 23° C.).

Polysorbate or polysorbate 20 may be added to a solution or load solution to a final concentration of 0.5% (% w/v) and the Polysorbate or polysorbate 20/(load) solution mix, incubated for 20±5 minutes at 23° C.±2° C.

As stated, a solution, for example a load solution, may be subject to some optimisation protocol before it is subjected to a purification (for example a chromatography) process. The parameters selected for optimisation may be those that have some effect on the efficiency and/or yield of a target protein from a protein/peptide purification process (into which the load solution is fed). Indeed a protocol for optimisation and the associated change in one or more parameters of the solution may lead to protein aggregation and/or clumping and ultimately, precipitation, within the load solution.

The pH of a load solution may be altered so that it is optimal for an affinity purification process. A pH which is optimal for a protein purification process may be sub-optimal for the solubility of some or all of the proteins in the load solution, including some or all of the target proteins. As such, altering the pH as part of an optimisation protocol may have the effect of causing one or more of the protein(s) or peptide(s) in the load solution to precipitate.

The optimal pH of a solution (or load solution) for use in a protein purification procedure, for example a chromatograph-based purification procedure, may vary depending on the nature of the solution, the protein to be purified and/or the specifics of the protein purification procedure to be used. For example, an optimal pH may be in the region of pH4, pH4.5. pH5, pH5.5, pH6, pH6.5, pH7, pH7.5, pH8, pH8.5, pH9 or pH9.5. Accordingly, optimisation may involve adjusting (by for example titration) a solution from one pH (for example a sub-optimal pH) to another, optimal pH.

The conductivity of a solution, for example a load solution, may also be altered so that it is optimal for an affinity purification processes. Conductivity refers to the specific conductance of the solution and is reported in units of millisiemens (mS). A conductivity which is optimal for a protein purification process may be sub-optimal for the solubility of some or all proteins in the load solution, including some or all of the target proteins. As such, altering the conductivity as part of an optimisation protocol may have the effect of causing one or more of the protein(s) or peptide(s) in the load solution to precipitate.

The optimal conductivity of a solution (or load solution) for use in a protein purification procedure, for example a chromatograph-based purification procedure, may vary depending on the nature of the solution, the protein to be purified and/or the specifics of the protein purification procedure to be used. For example, an optimal conductivity may be in the region of 8±2 mS/cm, 9±2 mS/cm, 10±2 mS/cm, 12±2 mS/cm, 14±2 mS/cm, 16±2 mS/cm, 18±2 mS/cm, 20±2 mS/cm, 22±2 mS/cm or 24±2 mS/cm. Accordingly, optimisation may involve adjusting a solution from one conductivity (for example a sub-optimal conductivity) to another, optimal conductivity.

While separately altering the pH and/or conductivity of a solution (for example a load solution) may bring about a level of protein/peptide precipitation, the effect of altering both pH and conductivity together may be significant and more of the protein(s) and/or peptide(s) in the solution may precipitate.

In view of the above, there is provided a method of preventing proteins in a load solution from precipitating, said method comprising contacting a load solution with an amount of polysorbate. Moreover, there is provided a method of preventing proteins in a load solution from precipitating, said method comprising contacting a load solution with an amount of polysorbate 20 (PS20).

It should be noted that the term ‘preventing’ may embrace any reduction in the instance of precipitation from a load solution versus the rate or amount of precipitation from a load solution not subjected to a method of this disclosure (i.e. not contacted with polysorbate/polysorbate 20 (PS20)).

The load solution (to which an amount of polysorbate or PS20 is added) may be optimised for an affinity chromatography process.

By way of example, the pH of the load solution may need to be optimised.

Additionally or alternatively, the conductivity of the load solution may need to be optimised.

The disclosed method may ensure that at least the target protein(s) or peptide(s) remain wholly or partly in solution immediately prior to use of the load solution in a protein purification process, for example an affinity chromatography process.

It should be noted that a solution, for example a load solution, to be subjected to a method of this disclosure may be contacted with an amount of polysorbate or polysorbate 20 (PS20: the amount being set out above) before, during or after an optimisation protocol has been (or is to be) applied to the solution. A method of this disclosure may further comprise contacting a solution with an amount of polysorbate/PS20 before the solution is applied, or subjected to, a purification process.

Accordingly, the disclosure provides a method of preventing target proteins in a load solution from precipitating in response to the optimisation of that load solution for use in a protein purification protocol, said method comprising contacting the load solution with an amount of polysorbate or polysorbate 20 (PS20) and then optimising the load solution for use in the protein purification protocol.

A method for preventing target protein precipitation in a load solution in response to the optimisation of that load solution for use in a protein purification protocol, may comprise contacting the load solution with polysorbate or polysorbate 20 (PS20) to a final concentration at any of the concentrations set out above. For example, the load solution may be contacted with polysorbate or PS20 at final concentration of 0.5% (% w/v) and then optimising the load solution for use in the protein purification protocol.

The step of contacting the load solution with polysorbate or PS20 (for example PS20 to a final concentration of 0.5% (% w/v)) may further include incubating the polysorbate or PS20/load solution mix for a period of time and at a suitable temperature to prevent protein (for example target protein) precipitation from the load solution. Suitable incubation times and temperatures are set out above and these apply here. For example, the polysorbate or PS20/load solution mix, may be incubated for 20±5 minutes at 23° C.±2° C.

Any of the methods of this disclosure may find application in methods of purifying glycoproteins. That is to say, a method for the prevention of protein precipitation described herein may be applied to solutions containing glycoproteins, which glycoproteins are for purification from the solution. The disclosed methods may be used to prevent (reduce or inhibit) the precipitation of those glycoproteins from the solution.

In some cases a load solution may comprise one or more glycoproteins. Indeed one or more of the glycoproteins within the load solution may be a (or the) target protein. That is, the load solution may comprise one or more glycoproteins that are to be purified from the load solution by, for example, a chromatography-based technique.

The term “glycoproteins” may embrace those proteins known as gonadotropins. For example, the term “glycoproteins” may include follicle stimulating hormone (FSH), human chorionic gonadotropin (hCG) and luteinising hormone (LH). Many glycoproteins, including the gonadotrophins, are used in therapeutic treatments. For example, gonadotropins (like FSH, LH and hCG) are used in the treatment of infertility.

It should be noted that the terms “glycoprotein”, “FSH” and hCG” embrace all isoforms and/or recombinant or naturally occurring forms thereof. The glycoproteins may be, for example, recombinant glycoproteins such as recombinant FSH, recombinant hCG or recombinant LH, produced in host cells. The glycoproteins may be provided in the form of a solution, from which solution they are to be purified or extracted by affinity chromatography.

In view of the above, the methods described herein may be applied to a solution, for example a load solution, comprising one or more protein(s) selected from the group consisting of:
(i) a glycoprotein;
(ii) FSH;
(iii) recombinant FSH (rFSH);
(iv) hCG; and
(v) recombinant hCG (rhCG);
wherein the one or more proteins (selected from the group consisting of proteins (i)-(v) above) are to be purified from the solution or load solution.

The proteins/glycoproteins may be urinary derived.

The glycoprotein may be present (in a solution, for example a load solution) as a single isoform or as a mixture of isoforms, as is well known in the art. Accordingly, a solution (or load solution) to be subjected to a method of purifying or extracting a target protein may comprise, for example, a quantity of a recombinant FSH, or a naturally occurring form of FSH (e.g. wherein FSH is present as a single isoform or as a mixture of isoforms).

The glycoprotein component of a solution may represent the target protein to be extracted and which is at risk of precipitation.

Thus, this disclosure provides a method of preventing glycoprotein precipitation from a solution, said method comprising contacting a solution comprising glycoproteins with an amount of polysorbate or polysorbate 20 (PS20).

Additionally, this disclosure provides a method of preventing glycoprotein precipitation from a solution in response to the optimisation of that solution for use in a glycoprotein purification protocol, said method comprising contacting the solution comprising a glycoprotein with an amount of polysorbate or polysorbate 20 (PS20) and then optimising the solution for use in the procedure to purify the glycoprotein.

As stated, the optimisation of a solution containing a glycoprotein may induce precipitation of that glycoprotein from the solution. To minimise the risk of ‘optimisation induced precipitation’ events, a solution comprising a glycoprotein for purification may comprise contacting the solution with polysorbate or polysorbate 20 (PS20) to a final concentration at any of the concentrations set out above. For example, the solution may be contacted with polysorbate or PS20 at final concentration of 0.5% (% w/v) the solution may then (or concurrently) be optimised for use in a protein purification protocol. The step of contacting the solution with polysorbate or PS20 (for example PS20 to a final concentration of 0.5% (% w/v)) may further include incubating the polysorbate or PS20/solution mix for a period of time and at a suitable temperature to prevent glycoprotein precipitation from the solution. Suitable incubation times and temperatures are set out above and these apply here. For example, the polysorbate or PS20/solution mix, may be incubated for 20±5 minutes at 23° C.±2° C. The solution may then be optimised as required and then fed into (or used in) a protein purification procedure.

Optimisation of a glycoprotein containing solution for use in a procedure for purifying one or more of the glycoproteins therein, may involve adjusting the pH and/or conductivity of the solution.

For example, the pH of a solution (for example a load solution) may be altered so that it is optimal for affinity purification of a glycoprotein solute. A pH which is optimal for a glycoprotein purification process may be sub-optimal for the solubility of some or all of the proteins, including the glycoproteins, in the solution/load solution. As such, altering the pH as part of an optimisation protocol may have the effect of causing one or more of the glycoproteins (and/or other protein(s) or peptide(s)) in the solution/load solution to precipitate.

The optimal pH of a solution (or load solution) for use in a protein purification procedure, for example a chromatograph-based purification procedure, may vary depending on the nature of the solution, the glycoprotein to be purified and/or the specifics of the glycoprotein purification procedure to be used. For example, for the purification of FSH and/or hCG from a solution (for example a load solution) an optimal pH may be in the region of pH4, pH4.5. pH5, pH5.5, pH6, pH6.5, pH7, pH7.5, pH8, pH8.5, pH9 or pH9.5. In one teaching, the optimal pH may be pH8. Accordingly, optimisation may involve adjusting (by for example titration) a solution containing a glycoprotein for purification, from one pH (for example a sub-optimal pH) to another, optimal pH.

The conductivity of a solution may also be altered so that it is optimal for affinity purification of a glycoprotein solute (as noted above, the term ‘conductivity’ refers to the specific conductance of the solution and is reported in units of millisiemens (mS)). A conductivity which is optimal for a glycoprotein purification process may be sub-optimal for the solubility of some or all proteins (including the glycoproteins) in the solution (or load solution). As such, while it may be necessary to alter the conductivity of a solution to ensure that it is optimal for the selected glycoprotein optimisation process, that optimisation may have the effect of causing one or more of the glycoproteins (or any of the other protein solutes) to precipitate.

The optimal conductivity of a solution (or load solution) for use in a glycoprotein purification procedure, for example a chromatograph-based purification procedure, may vary depending on the nature of the solution, the glycoprotein to be purified and/or the specifics of the protein purification procedure to be used. For example, an optimal conductivity may be in the region of 8±2 mS/cm, 9±2 mS/cm, 10±2 mS/cm, 12±2 mS/cm, 14±2 mS/cm, 16±2 mS/cm, 18±2 mS/cm, 20±2 mS/cm, 22±2 mS/cm or 24±2 mS/cm. In one teaching, the optimal conductivity may be 16±2 mS/cm. Accordingly, optimisation may involve adjusting a solution from one conductivity (for example a sub-optimal conductivity) to another, optimal conductivity.

While separately altering the pH and/or conductivity of a solution (for example a load solution) containing glycoproteins for purification may bring about a level of glycoprotein precipitation, the effect of altering both pH and conductivity together may be significant and more of the glycoprotein(s) in the solution may precipitate.

Where the glycoprotein is FSH and/or hCG (i.e. a solution comprises FSH and/or hCG for purification), the optimal pH may be in the region of pH8 and the optimal conductivity may be in the region of 16±2 mS/cm. Prior to optimisation, a solution from which a glycoprotein—for example FSH and/or hCG is to be purified, may have a sub-optimal (higher or lower than pH8) pH and/or (higher or lower than 16±2 mS/cm) conductivity.

The properties and composition of a solution, for example a load solution, comprising a glycoprotein for purification and/or extraction and to be subjected to a method of this disclosure, may vary depending on the source of the glycoprotein. Accordingly, the amount of type of optimisation required will also vary.

Where the glycoproteins are recombinant and generated or produced in a cell, a method of obtaining a solution or load solution comprising the recombinant glycoprotein (for example recombinant FSH and/or hCG) may comprise providing a cell which is capable of expressing a recombinant glycoprotein to be purified, culturing the cell in a suitable medium and then inducing the expression of the recombinant glycoprotein. The expressed glycoprotein may then be harvested from the cell (via a lysate prepared from the cell) and/or the medium (e.g. harvesting the recombinant protein from the cell culture supernatant by, for example, centrifugation). In this case, the solution or load solution may comprise or be derived from a culture medium.

Suitable methods of culturing transfected cells and inducing the expression of heterologous nucleic acids (or vectors) therein will be known and further information may be derived from, for example, ‘Production of Recombinant Proteins: Novel Microbial and Eukaryotic Expression Systems’: Edited by Prof. Dr. Gerd Gellissen; Pub. Wiley (2004).

Any suitable host cell may be used for the production of recombinant proteins and the recombinant glycoproteins described herein may be produced using, for example mammalian cells. Suitable mammalian cells (or cell lines) may include, for example CHO cells, PER.C6@ cells (information regarding the PER.C6@ cells is disclosed in PCT/EP2016/064668 (published as WO2016/207353)), HEK293 cells, HT1080 cell, COS cells, NOS cells, SP20 cells and the like.

The cell line may be cultured under suitable conditions and in a suitable medium and the target protein (for example a glycoprotein) may be harvested from the cell or from the culture medium.

A cell lysate and/or culture medium may be subject to a clarification or filtration protocol. A protocol of this type may use, for example, a depth filter. The clarification or filtration protocol may help purge or remove contaminants that could affect downstream processes. For example, the clarification and/or filtration process may help remove host cell proteins (HCP), DNA, potential lipids, colloids, cell debris, endotoxins and other material from the cell culture medium. A clarification or filtration step may further serve to protect the membranes used in a downstream filtration process which may be required to yield a useful load solution.

The filtered and/or clarified cell lysate/culture medium may then be further processed to concentrate any target protein content. The step of concentrating may be achieved through the use of an ultra-filtration step which is designed to further purge the filtered and/or clarified cell lysate culture medium of additional low molecular weight components (components which are less than <10Kda). An ultra-filtration process may be used to remove pigments and the like.

One of skill will appreciate that non-target protein content within a load solution, including for example any low molecular weight compounds or pigments may adversely affect protein purification or extraction process—in particular an affinity-chromatography based process.

The filtered/clarified and ultra-filtered cell medium may (optionally) be subject to a sterilisation procedure. For example, the filtered/clarified and ultra-filtered cell medium may be filtered so as to remove contaminants. The filtered/clarified and ultra-filtered cell medium may be passed through a 0.2 μm filter.

The concentration of the target protein (for example rFSH or rhCG) may be, for example 0.05-2.0 mg/ml. For example, the concentration of the target protein may be about 0.06 mg/ml, 0.07 mg/ml, 0.08 mg/ml, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, or about 1.9 mg/ml. The target protein concentration may be between about 0.4-1.5 mg/mL.

The total protein concentration may be about 1.0 mg/ml, 1.5 mg/ml, 1.75 mg/ml, 2.0 mg/ml, 2.25 mg/ml, 2.5 mg/ml, 2.75 mg/ml, 3.0 mg/ml, 3.25 mg/ml, 3.5 mg/ml, 3.75 mg/ml or 4.0 mg/ml. The filtered/clarified and ultra-filtered cell medium (which may be optionally sterlised as noted above) may comprise or be supplemented with additional buffers and/or excipients. For example, the filtered/clarified and ultra-filtered cell medium may comprise or be supplemented with glycine (or example 100 mM glycine) and/or NaCl (for example 50 mM NaCl). The filtered/clarified and ultra-filtered cell medium may have a pH of about 9.0±0.2, and a conductivity of about 6±1 mS/cm, or about 7±1 mS/cm, or about 8±1 mS/cm, or about 9±1 mS/cm, or about 10±1 mS/cm.

The filtered/clarified and ultra-filtered cell medium (which may be optionally sterlised as noted above) may be used to prepare a load solution.

A load solution may be prepared by combining a volume of the above described (and optionally sterilised) filtered/clarified and ultra-filtered cell medium with a volume of another ‘load stock solution’.

The load stock solution may comprise, for example certain excipients, diluents and/or buffers. For example, the load stock solution may comprise an amount of Tris and NaCl. For example, the load stock solution may comprise 0.8M Tris and 3M NaCl. The load stock solution may have a pH of about 8.3—for example pH8.3±0.2. The load stock solution may have a conductivity of about 164 mS/cm, e.g. 164±2 mS/cm.

A volume, for example 40 ml of the load stock solution may be added to every liter of the above described (and optionally sterilised) filtered/clarified and ultra-filtered cell medium to provide a combined solution which is a load solution. The pH of the resulting load solution may be adjusted to pH 8.0±0.2 with, for example, 1 M HCl. The conductivity of the load solution may be 16±2 mS/cm. A solution/load solution prepared and containing glycoproteins for purification, may be optimised so that the load solution may be used in a process designed to purify the target (recombinant) glycoprotein. As stated, the act of optimising a load solution of this type (by adjustment of one or more of the parameters of the solution) may induce protein precipitation (including glycoprotein precipitation) which may adversely affect the yield and purity of the protein purification procedure. The methods described herein, which methods contact the solution or load solution (before, during or after an optimisation protocol) with an amount of PS20, can prevent, inhibit and/or reduce those precipitation events.

It should be noted that the pH and/or conductivity any solution or load solution, including those comprising (recombinant) glycoproteins for purification, may be determined and any optimisation (to bring the pH and/or conductivity to within accepted and optimal parameters) applied. Prior to implementation of any determined optimisation requirement, the solution may be contacted with an amount of PS20 to minimise the risk of an optimisation induced precipitation event.

In view of the above, a method of purifying or extracting a recombinant protein, may comprise:
obtaining or providing a solution comprising the recombinant protein
adding polysorbate or polysorbate 20 to the solution; and
loading the solution on to an affinity chromatography column designed to purify or extract the recombinant protein.

The recombinant protein may be recombinant FSH or recombinant hCG.

The method may further comprise a step in which the pH and/or conductivity of the solution is/are determined. If the pH and/or conductivity of the solution are determined to be sub optimal, an amount of polysorbate or PS20 may be added and then the solution optimised so that the pH and/or conductivity are optimal.

Methods for making recombinant glycoproteins—including, for example recombinant FSH and recombinant hCG are described in WO2009/127826, WO2013/020996, WO2016207353 and WO2011042688 and the entire contents of all of these documents is incorporated herein by reference. To the extent these methods use protein purification procedures, for example chromatography and/or affinity chromatography procedures, to extract recombinant FSH and/or hCG, any load solutions to be used may be subject to a method of this invention. For example, a load solution may be contacted with a disclosed amount of PS20 so that it can be optimised for use in a purification procedure, minimising the risk that that optimisation procedure induces a (FSH and/or hCG) precipitation event.

Disclosed herein is a method of preventing, reducing or inhibiting optimisation induced precipitation of FSH and/or hCG from a solution, said method comprising contacting the solution with an amount of PS20. The amount of PS20 is sufficient to retain the FSH and/or hCG in solution throughout an optimisation protocol.

One of skill will appreciate that purified proteins may be present in one or more of the fractions collected from a chromatography process. The amount of target protein in any given fraction may vary, with some fractions containing more target protein than others. To increase the amount of target protein collected, fractions may be pooled. Collected and/or pooled fractions may be subject to additional purification and/or concentrating protocols.

Fractions obtained from the protein purification process (for example an affinity chromatography process) may be further processed by pooling, to combine fractions comprising the target protein(s) and/or to increase the concentration of the target protein(s).

### DETAILED DESCRIPTION

The present invention will now be described with reference to the following examples.

### Example 1

### PS20 Optimization Experiment to Achieve the Optimal Conditions

Prior to the demonstration that QS chromatography has the capability to remove potential enveloped-viruses, there is a dedicated step for inactivation of enveloped viruses in which Harvest-AD was added with PS20 to a final conc. of 1% (% w/v) followed by 1 h incubation at 23±2° C. Please note, the term ‘Harvest-AD’ generally embraces material harvested from a protein purification/extraction protocol. This material may comprise a load solution as defined herein (the load solution comprising one or more target proteins for purification/extraction). In one embodiment ‘Harvest-AD’ is the material yielded from any ultrafiltration/diafiltration step(s)—the term ‘AD’ refers to ‘after diafiltration’.

### PS20 as a Solubilizer:

An affinity load (or ‘load solution’) containing, for example, rhCG may be loaded at pH 8 and with a conductivity of 16±2 mS/cm. A lower conductivity may allow non-specific proteins to bind to the resin and block the binding sites to rhCG; if that non-specific binding is irreversible a decrease in rhCG capacity or yield will appear over time.

Harvest-AD from which the Affinity-Load may be prepared at pH 9.0 and with a conductivity of ˜5 mS/cm.

When Harvest-AD pH was titrated to 8.0 and conductivity raised to 16±2 mS/cm (=Affinity-Load), without PS20 (to, for example, a final concentration of 1% (% w/v)), protein precipitation appeared (see results below).

When Harvest-AD was added with PS20 to a final concentration of 1% (% w/v) and incubated for 1 h at 23±2° C. prior to pH & conductivity adjustment, there was no precipitation. Therefore PS20 helps prevent protein precipitation in the Affinity Load.

Experiments were carried out to find the minimum PS20 concentration that is required to obtain a stabilized Affinity Load (pH 8.0 & 16±2 mS/cm).

After finding that the addition of PS20 to a final concentration of 0.5% (% w/v) yields a stable Affinity Load, the incubation time was reduced from 1 h to 20 min and no precipitation appeared in the Affinity Load (Data not shown)

However, PS20 generates radicals that may increase target protein oxidation (in this example the oxidation of rhCG). Accordingly, reducing the PS20 concentration and incubation time will lower the risk of such oxidation events.

Experimentation showed that PS20 to a final concentration of 0.5% (% w/v) and an incubation time/temperature of 20±5 min at 23±2° C. helped reduce precipitation of target proteins (for example rhCG) from a load solution and reduce the risk of oxidation events caused by the addition of PS20.

H-AD: Is a rhCG-containing Harvest that is volumetrically-concentrated by an average of 50-fold (Min-Max range between 37-66-fold) containing an average of rhCG concentration of 0.8 mg/mL (Min-Max range between 0.4-1.5 mg/mL) and an average of total-protein conc, (acc. Bradford) of 3 mg/mL (Min-Max range between 2-5 mg/mL). H-AD diluent composed of 100 mM Glycine, 50 mM NaCl pH 9.0±0.2, 6±1 mS/cm.

Experiment 1

| | Step 1 | | | | | | | |
|---|---|---|---|---|---|---|---|---|
| | rhCG containing Harvest: [pH 7.2, 13.5 mS/cm]: (20 μg/mL rhCG, 163 μg/mL Bradford) | | | | | | | |
| | Step 2 | | | | | | | |
| | Harvest Clarification by positively charged Depth filter | | | | | | | |
| | Step 3 | | | | | | | |
| | 10 kDa UF1(Ultrafiltration) | | | | | | | |
| | Step 4 | | | | | | | |
| | Harvest-AD (=H-AD) in 100 mM Glycine + 50 mM NaCl pH 9.13, 5.06 mS/cm: | | | | | | | |
| | Volume: 3.257 L, A₂₈₀: 5.208, Bradford: 4.93 mg/mL, rhCG: 0.910 mg/ml | | | | | | | |
| | 1 | 2 | 3 | 4 | 5 | 6 | 7 | 8 |
| Step 4a | 40 mL H-AD | 40 mL H-AD | 40 mL H-AD | 40 mL H-AD | 40 mL H-AD | 40 mL H-AD | 40 mL H-AD | 40 mL H-AD |
| Step 4b | Add 40 mL | ↓ | ↓ | ↓ | ↓ | ↓ | Add 5M NaCl. | Add 5M NaCl. |
| | of[0.8M Tris, 3M | | | | | | Adjust to pH | Adjust to pH |
| | NaCl] sol. per | | | | | | 9.0 & 16 mS | 8.5 & 16 mS |
| | 1L H-AD. | | | | | | [titrate pH w/ | [titrate pH w/ |
| | Adjust to pH | | | | | | 1M HCl] | 1M HCl] |
| | 8.0 & 16 mS | | | | | | | |
| Step 4c | ↓ | Add 20% PS20 | Add 20% PS20 | Add 20% PS20 | Add 20% PS20 | Add 20% PS20 | ↓ | ↓ |
| | | to a final of | to a final of | to a final of | to a final of | to a final of | | |
| | | 0.1% PS20 | 0.25% PS20 | 0.5% PS20 | 0.75% PS20 | 1% PS20 | | |
| | | incu. 1 hr, | incu. 1 hr, | incu. 1 hr, | incu. 1 hr, | incu. 1 hr, | | |
| | | 23° C., w/stir | 23° C., w/stir | 23° C., w/stir | 23° C., w/stir | 23° C., w/stir | | |
| Step 4d | ↓ | Add 40 mL of | Add 40 mL of | Add 40 mL of | Add 40 mL of | Add 40 mL of | ↓ | ↓ |
| | | [0.8M Tris | [0.8M Tris | [0.8M Tris | [0.8M Tris | [0.8M Tris | | |
| | | 3M NaCl] sol. | 3M NaCl] sol. | 3M NaCl] sol. | 3M NaCl] sol. | 3M NaCl] sol. | | |
| | | per 1 L H-AD | per 1 L H-AD | per 1 L H-AD | per 1 L H-AD | per 1 L H-AD | | |
| | | Adjust to pH | Adjust to pH | Adjust to pH | Adjust to pH | Adjust to pH | | |
| | | 8.0 & 16 mS | 8.0 & 16 mS | 8.0 & 16 mS | 8.0 & 16 mS | 8.0 & 16 mS | | |
| | | [titrate pH | [titrate pH | [titrate pH | [titrate pH | [titrate pH | | |
| | | w/1M HCl] | w/1M HCl] | w/1M HCl] | w/1M HCl] | w/1M HCl] | | |
| Step 4e | Check clarity by | Check clarity by | Check clarity by | Check clarity by | Check clarity by | Check clarity by | Check clarity by | Check clarity by |
| | VI and trans. | VI and trans. | VI and trans. | VI and trans. | VI and trans. | VI and trans. | VI and trans. | VI and trans. |
| | @ 660 nm | @ 660 nm | @ 660 nm | @ 660 nm | @ 660 nm | @ 660 nm | @ 660 nm | @ 660 nm |
| | At: | At: | At: | At: | At: | At: | At: | At: |
| | 1) Time zero | 1) Time zero | 1) Time zero | 1) Time zero | 1) Time zero | 1) Time zero | 1) Time zero | 1) Time zero |
| | 2) After 8 h at | 2) After 8 h at | 2) After 8 h at | 2) After 8 h at | 2) After 8 h at | 2) After 8 h at | 2) After 8 h at | 2) After 8 h at |
| | R.T | R.T | R.T | R.T | R.T | R.T | R.T | R.T |
| | 3) O.N incu. at | 3) O.N incu. at | 3) O.N incu. at | 3) O.N incu. at | 3) O.N incu. at | 3) O.N incu. at | 3) O.N incu. at | 3) O.N incu. at |
| | 4° C. | 4° C. | 4° C. | 4° C. | 4° C. | 4° C. | 4° C. | 4° C. |

*Abbreviations:
sol.: solution;
incu.: incubation;
VI: Visual inspection;
R.T: Room temperature;
O.N: Overnight;
trans: Transmission
The preparation of the Affinity Load at the final process is:
At the end of PS20 treatment (0.5% PS20, incubated with stirring for 20 ± 5 min at 23 ± 2° C.) 40 mL from solution [0.8M Tris, 3M NaCl pH 8.3 ± 0.2, 164 ± 2 mS/cm] is added per 1 L of Harvest-AD and the pH is adjusted to 8.0 ± 0.2 with 1M HCl.
Verifying that the obtained conductivity in Affinity Load is within 16 ± 2 mS/cm (the added volume of 0.8M Tris, 3M NaCl solution is designed to be within this conductivity range)

| | | | Final | | | | | % |
|---|---|---|---|---|---|---|---|---|
| | | | parameters | | Clarity of solution | | | Trans- |
| | | H-AD | | Final | | | After | mission |
| | | Volume | Final | Conductivity | At | After | O.N at | at |
| Sample name | | (mL) | pH | (mS/cm) | Time 0 | 8 h, RT | 4° C. | 660 nm |
| 0. | H-AD as is pH 9.0, ~5 mS/cm | 40 | NR | | Clear | Clear | Clear | 99.50 |
| 1. | H-AD pH9.0, ~5 mS/cm → to pH 8 & | 40 | 8.00 | 16.25 | Slightly | Slightly | Slightly | 35.38 |
| | 16 mS/cm, wo/PS20 | | | | white++ | white+++ | white+++ | |
| 2. | H-AD →Add final 0.1% PS20 → 1 h, stir 23 ± 2° C. → | 40 | 7.93 | 16.25 | Clear | Clear | Very | 96.11 |
| | to pH 8 ± 0.2 & 16 ± 2 mS/cm | | | | | | slightly | |
| | | | | | | | white+ | |
| 3. | H-AD →Add final 0.25% PS20 → 1 h, stir 23 ± 2° C. → | 40 | 8.02 | 16.10 | Clear | Clear | confuse | 96.63 |
| | to pH 8 ± 0.2 & 16 ± 2 mS/cm | | | | | | | |
| 4. | H-AD→ Add final 0.5% PS20 →1 h, stir 23 ± 2° C. | 40 | 8.02 | 15.77 | Clear | Clear | clear | 98.59 |
| | to pH 8 ± 0.2 & 16 ± 2 mS/cm | | | | | | | |
| 5. | H-AD →Add final 0.75% PS20 →1 h, stir 23 ± 2° C. → | 40 | 8.03 | 15.70 | Clear | Clear | clear | 98.18 |
| | to pH 8 ± 0.2 & 16 ± 2 mS/cm | | | | | | | |
| 6. | H-AD →Add final 1.0% PS20 →1 h, stir 23 ± 2° C. → | 40 | 8.01 | 15.75 | Clear | Clear | clear | 98.03 |
| | to pH 8 ± 0.2 & 16 ± 2 mS/cm | | | | | | | |
| 7. | H-AD pH 9.0, ~5 mS/cm → to 16 mS/cm, wo/PS20 | 40 | 9.05 | 15.90 | Clear | Clear | Slightly | 97.40 |
| | | | | | | | white+ | |
| 8. | H-AD pH 8.5, ~5 mS/cm → to 16 mS/cm, wo/PS20 | 40 | 8.50 | 15.70 | Slightly | Slightly | Slightly | 78.89 |
| | | | | | white+ | white++ | white++ | |

Note:
In Experiment 1: rhCG conc. in H-AD is 0.91 mg/mL and total-protein conc. (Bradford) is 4.9 mg/mL.

Experiment 2

| Step 1 | rhCG containing Harvest | | | |
|---|---|---|---|---|
| | [pH 7.2, 13.5 mS/cm] | | | |
| | (20 μg/mL rhCG, 163 μg/mL Bradford) | | | |
| Step 2 | Harvest Clarification by positively charged Depth filter | | | |
| Step 3 | 10 kDa UF1(Ultrafiltration) | | | |
| Step 4 | Harvest-AD (=H-AD) | | | |
| | in 100 mM Glycine + 50 mM NaCl pH 9.13, 5.06 mS/cm | | | |
| | Volume: 3.257 L, A₂₈₀: 5.208, Bradford: 4.93 mg/mL, rhCG: 0.910 mg/mL | | | |
| Step 4a | 40 mL H-AD | 40 mL H-AD | 40 mL H-AD | 40 mL H-AD |
| Step 4b | Add 5M NaCl | Add 5M NaCl | Add 5M NaCl | ↓ |
| | Adjust to pH 8.5 & | Adjust to pH 8.5 & | Adjust to pH 8.5 & | |
| | 10 mS | 12 mS | 15 mS | |
| | [titrate pH w/1M HCl] | [titrate pH w/1M HCl] | [titrate pH w/1M HCl] | |
| Step 4c | ↓ | ↓ | ↓ | Add 20% PS20 |
| | | | | to final of 0.4% PS20 |
| | | | | incu. 1 hr, 23º C., w/stir |
| Step 4d | ↓ | ↓ | ↓ | Add 40 mL of [0.8M Tris 3M |
| | | | | NaCl] sol. per 1 L H-AD |
| | | | | Adjust to pH 8.0 & 16 mS |
| | | | | [titrate pH w/1M HCl] |
| Step 4e | Check clarity by VI | Check clarity by VI | Check clarity by VI | Check clarity by VI |
| | and trans. at 660 nm | and trans. at 660 nm | and trans. at 660 nm | and trans. at 660 nm |
| | At: | At: | At: | At: |
| | Time zero | Time zero | Time zero | Time zero |
| | After 8 h at R.T | After 8 h at R.T | After 8 h at R.T | After 8 h at R.T |
| | O.N incu. at 4º C. | O.N incu. at 4° C. | O.N incu. at 4º C. | O.N incu. at 4º C. |

* Abbreviations: sol.: solution; incu.: incubation; VI: Visual inspection; R.T: Room temperature; O.N: Overnight; trans: Transmission

In Experiment 2: rhCG conc. in H-AD is 0.91 mg/mL and total-protein conc. (Bradford) is 4.9 mg/mL.

| | | | | | | | | % |
|---|---|---|---|---|---|---|---|---|
| | | | Final Parameters | | Clarity of solution | | | Trans- |
| | | H-AD | | Final | At | After | After | mission |
| | | volume | Final | Conductivity | Time | 8 h, | O.N at | at |
| Sample name | | (mL) | pH | (mS/cm) | 0 | RT | 4° C. | 660 nm |
| 0. | H-AD as is pH 9.0, ~5 mS/cm | 40 | NR | | clear | clear | clear | 99.0 |
| 1. | H-AD pH 9.0, ~5 mS/cm → | 40 | 8.50 | 10.00 | clear | Slightly | Slightly | 97.8 |
| | to pH 8.5 & 10 mS/cm, wo/PS20 | | | | | white+ | white+ | |
| 2. | H-AD pH 9.0, ~5 mS/cm → | 40 | 8.47 | 12.00 | clear | Slightly | Slightly | 97.2 |
| | to pH 8.5 & 12 mS/cm, wo/PS20 | | | | | white+ | white+ | |
| 3. | H-AD pH 9.0, ~5 mS/cm → | 40 | 8.50 | 15.20 | Slightly | Slightly | Slightly | 95.9 |
| | to pH 8.5 & 15 mS/cm, wo/PS20 | | | | white+ | white++ | white+++ | |
| 4. | H-AD Add final 0.4% PS20 → | 40 | 8.00 | 16.20 | Clear | Clear | Clear | 98.2 |
| | 1 h, stir 23 ± 2° C. to pH 8 & 16 mS/cm | | | | | | | |

Experiment 3

| Step 1 | rhCG containing Harvest; [pH 7.2, 13.5 mS/cm](20 μg/mL rhCG, 163 μg/mL Bradford) | |
|---|---|---|
| Step 2 | Harvest Clarification by positively charged Depth filter | |
| Step 3 | 10 kDa UF1(Ultrafiltration) | |
| Step 4a | 220 mL Harvest-AD | 220 mL Harvest-AD |
| Step 4b | Add 20% PS20 | Add 20% PS20 |
| | to final of 0.25% PS20 | to final of 0.5% PS20 |
| | incu. 1 hr, 23º C., w/stir | incu. 1 hr, 23º C., w/stir |
| Step 4c | Add 40 mL of [0.8M Tris, | Add 40 mL of [0.8M Tris, |
| | 3M NaCl] sol. per 1 L H-AD | 3M NaCl] sol. per 1 L H-AD |
| | Adjust to pH 8.0 & 16 mS | Adjust to pH 8.0 & 16 mS |
| | [titrate pH w/1M HCl] | [titrate pH w/1M HCl] |
| Step 4d | Check clarity by V and trans. at 660 nm | Check clarity by VI and trans. at 660 nm |
| | At: | At: |
| | Time zero | Time zero |
| | After 8 h at R.T | After 8 h at R.T |
| | O.N incu. at 4º C. | O.N incu. at 4º C. |

* Abbreviations: sol.: solution; incu.: incubation; VI: Visual inspection; R.T: Room temperature; O.N: Overnight; trans: Transmission

In Experiment 3: rhCG conc. in H-AD is 0.91mg/mL and total-protein conc. (Bradford) is 4.9 mg/mL.

| | | | | | | | | % |
|---|---|---|---|---|---|---|---|---|
| | | | Final parameters | | Clarity of solution | | | Trans- |
| | | H-AD | | Final | At | After | After | mission |
| | | Volume | Final | Conductivity | Time | 8 h, | O.N | at |
| Sample Name | | (mL) | pH | (mS/cm) | 0 | RT | at 4° C. | 660 nm |
| 0. | H-AD as is pH 9.0, ~5 mS/cm | 40 | NR | | Clear | Clear | Clear | 99.0 |
| 1. | A-AD Add final 0.25% PS20 →1 h, | 220 | 8.01 | 16.12 | Very | Slightly | Slightly | 98.0 |
| | stir 23 ± 2° C.→ to pH 8 & 16 mS/cm | | | | Slightly | white+ | white+ | |
| | | | | | white+ | | but some | |
| | | | | | | | precp. | |
| | | | | | | | Appears | |
| 2. | H-AD Add final 0.5% PS20 →1 h, | 220 | 8.03 | 15.90 | Clear | Clear | Clear but | 98.9 |
| | stir 23 ± 2° C. →to pH 8 & 16 mS/cm | | | | | | some | |
| | | | | | | | precp. | |
| | | | | | | | Appears | |

### Purification Runs

H-AD: is the rhCG-containing Harvest that is volumetrically-concentrated by an average of 50-fold by UF1 (Min-Max range between 37-66-fold) containing an average of rhCG concentration of 0.8 mg/mL (Min-Max range between 0.4-1.5 mg/mL) and an average of total-protein conc. (acc. Bradford) of 3 mg/mL (Min-Max range between 2-5 mg/mL).

H-AD diluent composed of 100 mM Glycine, 50 mM NaCl pH 9.0±0.2, 6±1 mS/cm [finally changed to 100 mM Glycine, 50 mM NaCl with same pH and conductivity].

### Preparation of Load Solution

After PS20 treatment (0.5% PS20, incubated with stirring for 20±5 min at 23±2° C.), 40 mL of a solution comprising 0.8M Tris, 3M NaCl pH 8.3±0.2, 164±2 mS/cm, is added per 1 L Harvest-AD. The pH is adjusted to 8.0±0.2 with 1 M HCl. The obtained conductivity in the affinity load solution is verified as being within 16±2 mS/cm (the added volume of 0.8M Tris, 3M NaCl solution is designed such to be within this conductivity range).

### Example 3: FSH Manufacturing Flow Chart

| Process Step | | Step Purpose |
|---|---|---|
| Thaw vial of rFSH WCB | | Cell growth prior to seeding |
| Cell propagation | | |
| (shake flasks and Wave | | |
| bioreactor) | | |
| Seeding and cell growth in 50 L | | rFSH production in the required quantity and quality. |
| BR | | |
| rFSH production and continuous | | |
| harvest using microfiltration | | |
| (ATF 10) system | | |
| Inline harvest clarification with | | Removal of host cell proteins (HCP), DNA, potential lipids and |
| depth filter (PDE2) | | colloids, cell debris and endotoxins. Provides protection for |
| | | UF1 membranes |
| 10 KDa UF 1 | | Concentrate rFSH to reduce the Affinity chromatography |
| | | process-time; removal of pigments and low molecular weight |
| | | components (<10 KDa) that may adversely affect the Affinity |
| | | resin |
| Bioburden control | | Bioburden control |
| Capture | Affinity | Load treatment: 0.5% (% w/v) polysorbate 20 (PS 20) addition |
| | chromatography | to increase solubility prior to pH adjustment. |

Chromatography: Capture of rFSH and removal of: HCP, LMW
subunits, PS 20, endotoxins and host cell DNA.

### Example 4: hCG Manufacturing Flow Chart

| Process Step | | Step Purpose |
|---|---|---|
| Thaw vial of rhCG WCB | | Cell growth prior to seeding |
| Cell propagation | | |
| (shake flasks and Wave | | |
| bioreactor) | | |
| Seeding and cell growth in 50 L | | rhCG production in the required quantity and quality. |
| BR | | |
| rhCG production and continuous | | |
| harvest using microfiltration | | |
| (ATF 10) system | | |
| Inline harvest clarification with | | Removal of host cell proteins (HCP), DNA, potential lipids |
| depth filter (PDE2) | | and colloids, cell debris and endotoxins. Provides protection |
| | | for UF1 membranes |
| Concentrate rhCG harvest to | | Concentrate rhCG to reduce the Affinity chromatography |
| reduce the Affinity | | process-time; removal of pigments and low molecular weight |
| chromatography process-time; | | components (<10 KDa) that may adversely affect the Affinity |
| removal of pigments and low | | resin |
| molecular weight components | | |
| (<10 KDa) that may adversely | | |
| affect the Affinity resin | | |
| Bioburden control | | Bioburden control |
| Capture | Affinity | Load treatment: 0.5% (% w/v) polysorbate 20 (PS 20) addition |
| | chromatography | to increase solubility prior to pH adjustment. |

Chromatography: Capture of rhCG and removal of: HCP,
ß-hCG subunits, PS 20, endotoxins and host cell DNA.

### Example 5

For rFSH preparation, the process and techniques developed for rhCG preparation were applied.

rFSH harvest using 0.5% or 1% (% w/v) PS20 yielded a clear solution (with no precipitate).
Volumetrically-concentrated, average of 39-fold (Min-Max range between 34-47-fold) with an average of rFSH concentration of 0.09 mg/mL (Min-Max range between 0.07-0.138 mg/mL)
An average of total-protein conc. (acc. Bradford) of 1.3 mg/mL (Min-Max range between 0.7-1.8 mg/mL).
H-AD diluent composed of 100 m M Glycine, 60 mM NaCl pH 9.0±0.2, 9.0±0.5 mS/cm
   The present invention is further described by reference to the following numbered clauses:

1. A method of preventing protein precipitation from a solution, said method comprising contacting the solution with an amount of polysorbate 20 (PS20).

2. A method of preventing protein precipitation from a solution, said method comprising contacting the solution with an amount of polysorbate.

3. The method of clause 2, wherein the polysorbate is selected from the group consisting of polysorbate 20 (PS20); polysorbate 40 (PS40); polysorbate 60 (PS60); and polysorbate 80 (PS80).

4. The method of clauses 1-3, wherein the solution is for use in, or with, a protein purification process.

5. The method of clauses 1-4, wherein the solution is a load solution, for use with a protein purification process.

6. The method of clauses 4-5, wherein the protein purification process comprises affinity chromatography.

7. The method of any preceding clause wherein the solution comprises one or more proteins selected from the group consisting of:
(i) a glycoprotein or a recombinant glycoprotein;
(iii) FSH or recombinant FSH; and
(iv) hCG or recombinant hCG.

8. The method of any preceding clause wherein the protein purification process is for the purification of a protein selected from the group consisting of:
(i) a glycoprotein or a recombinant glycoprotein;
(iii) FSH or recombinant FSH; and
(iv) hCG or recombinant hCG.

9. The method of any one of clauses 1 and 4-8, wherein the amount of PS20 is an amount yielding a final concentration of PS20 in the solution of between about 0.1% (% w/v) and 5% (% w/v).

10. The method of any one of clauses 1 and 4-9, wherein the amount of PS20 is an amount yielding a final concentration of PS20 in the solution of 0.5% (% w/v).

11. The method of any one of clauses 1 and 4-10, wherein the PS20 is contacted with the solution for anywhere between about 1 minute and 2 hours.

12. The method of any one of clauses 1 and 4-11, wherein the PS20 is contacted with the solution for 15-25 minutes.

13. The method of any one of clauses 1 and 4-12, wherein the PS20 is contacted with the solution at a temperature of anywhere between about 10° C. and 30° C.

14. The method of any one of clauses 1 and 4-13, wherein the PS20 is contacted with the solution at a temperature of anywhere between about 21° C. and 25° C.

15. The method of any one of clauses 1 and 4-14, wherein after contact with PS20, the solution or load solution is optimised for use with a protein purification process.

16. The method of clause 15, wherein the optimisation of the solution or load solution involves adjusting the pH and/or conductivity.

17. The method of clauses 15 or 16, wherein the solution is prepared at pH8 and with a conductivity of between 14 and 18 mS/cm.

18. The method of any preceding clause, wherein the solution is derived from a cell culture medium.

19. The method of clause 18, wherein prior to contact with PS20, host cell proteins, DNA, potential lipids, colloids, cell debris and/or endotoxins are removed the solution derived from the cell culture medium.

20. A method of preventing protein precipitation during the optimisation of a load solution for affinity chromatography, said method comprising:
contacting a load solution to be optimised for affinity chromatography with an amount of PS20 to provide a PS20/load solution; and
optimising the PS20/load solution mix for affinity chromatography.

21. The method of clause 20, wherein the amount of PS20 contacted with the load solution yields a load solution with a final concentration of PS20 of between about 0.1% (% w/v) and 5%.

22. The method of clauses 20 or 21 wherein the PS20/load solution mix is incubated from anywhere between 15-25 minutes and at a temperature of between 21° C. and 25° C.

23. The method of clauses 20-22, wherein the load solution is derived from a cell culture medium and contains a recombinant glycoprotein.

24. The method of clauses 20-23, wherein the load solution is derived from a cell culture medium and contains recombinant FSH or recombinant hCG.

25. A method of preparing a load solution for use with an affinity chromatography process; said method comprising contacting the load solution with an amount of PS20 to provide a PS20/load solution mix.

26. The method of clause 25, wherein the PS20/load solution mix comprises between 0.1% and 5% (% w/v) PS20.

27. The method of clause 25 or 26, wherein the PS20/solution mix is incubated for anywhere between 15-25 minutes and at a temperature of between 21° C. and 25° C.

28. The method of clauses 25-27, wherein the load solution is derived from a cell culture medium and contains a recombinant glycoprotein.

29. The method of clauses 25-28, wherein the load solution is derived from a cell culture medium and contains recombinant FSH or recombinant hCG.

30. The method of anyone of clauses 25-29, wherein after contact with PS20, the load solution is optimised for use with a protein purification process.

31. The method of clause 30, wherein the optimisation of the load solution involves adjusting the pH and/or conductivity of the load solution.

32. The method of clauses 30 to 31, wherein the load solution is prepared at pH8 and with a conductivity of between 14 and 18 mS/cm.

33. A method of purifying a protein, said method comprising:
(a) obtaining or providing a solution containing a protein to be purified;
(b) contacting the solution with an amount of PS20; and
(c) purifying the protein from the solution.

34. The method of clause 33 wherein the protein to be purified is a protein selected from the group consisting of:
(i) a glycoprotein or a recombinant glycoprotein;
(ii) FSH or recombinant FSH; and
(iii) hCG or recombinant hCG.

35. The method of clauses 33 and 34, wherein the solution is derived from a cell culture medium.

36. The method of clause 35 wherein the solution is clarified and/or filtered before being contacted with PS20.

37. The method of clause 36 wherein host cell proteins, DNA, potential lipids and colloids, cell debris and endotoxins are removed from the solution before it is contacted with PS20.

38. The method of any one of clauses 33-37, wherein the protein is purified by affinity chromatography.

39. The method of any one of clauses 33-38, wherein the purified protein is eluted and collected.

40. The method of clause 39, wherein the eluted and collected protein is not treated with a combination of caprylic acid and ethanol.

41. The method of any one of clauses 39 and 40, wherein the eluted and collected protein is not filtered using a glass fibre filter.

42. The method of any one of clauses 39-41, wherein the eluted and collected protein is not subject to sulfopropyl sepharose chromatography and/or hydrophobic interaction chromatography.

1-16. (canceled)

17. A method of preventing protein precipitation from a load solution, said method comprising contacting the solution with an amount of polysorbate 20 (PS20).

18. The method of claim 17, wherein the load solution is a load solution for a protein purification process.

19. The method of claim 17, wherein the load solution is an affinity chromatography load solution.

20. The method of claim 17, wherein the method prevents precipitation from the solution of one or more proteins selected from:
(i) naturally-occurring glycoproteins and recombinant glycoproteins;
(iii) naturally-occurring FSH and recombinant FSH; and
(iv) naturally-occurring hCG and recombinant hCG.

21. The method of claim 17, wherein the amount of PS20 yields a final concentration of PS20 in the solution of between about 0.1% w/v and 5% w/v.

22. The method of claim 17, wherein the amount of PS20 yields a final concentration of PS20 in the solution of 0.5% w/v.

23. The method of claim 17, wherein the PS20 is contacted with the solution for from 1 minute to 2 hours.

24. The method of claim 17, wherein the PS20 is contacted with the solution for 15-25 minutes.

25. The method of claim 17, wherein the PS20 is contacted with the solution at a temperature from about 10° C. to 30° C., optionally wherein the PS20 is contacted with the solution at a temperature from about 21° C. to 25° C.

26. The method of claim 17, wherein the solution is contacted with PS20 at a final concentration of 0.5% w/v PS20 for 20±5 min at 23±2° C.

27. The method of claim 17, further comprising, after the contacting with PS20, adjusting the solution to have a pH of 8 and a conductivity of between 14 and 18 mS/cm.

28. A method of preventing protein precipitation during optimization of a load solution for affinity chromatography, said method comprising:
contacting the a load solution with an amount of PS20 to provide a PS20/load solution; and
optimizing the PS20/load solution for affinity chromatography.

29. The method of claim 28, wherein the amount of PS20 contacted with the load solution yields a load solution with a final concentration of PS20 of between about 0.1% w/v and 5% w/v.

30. The method of claim 28, wherein the PS20/load solution is incubated for 15-25 minutes at a temperature from 21° C. to 25° C.

31. The method of claim 28, wherein the load solution comprises recombinant FSH or recombinant hCG.

32. A method of purifying a protein, said method comprising:
(a) contacting a solution containing a protein to be purified with an amount of PS20; and
(b) purifying the protein from the solution.

33. The method of claim 32, wherein the protein to be purified is selected from:
(i) naturally-occurring glycoproteins and recombinant glycoproteins;
(ii) naturally-occurring FSH and recombinant FSH; and
(iii) naturally-occurring and recombinant hCG.

34. The method of claim 32, wherein the method comprises contacting the solution with PS20 at a final concentration of 0.5% w/v PS20 for 20±5 min at 23±2° C.
